(19) United States
(12) Patent Application Publication
(10) Pub. No.: US 2010/0234288 A1
(43) Pub. Date: Sep. 16, 2010

(54) SUSTAINED RELEASE PHARMACEUTICAL COMPOSITION ON THE BASIS OF RELEASE SYSTEM COMPRISING AN ACID-SOLUBLE POLYMER AND A pH-DEPENDENT POLYMER

(75) Inventors: Rajesh Jain, New Delhi (IN); Kour Chand Jindal, New Delhi (IN); Sampath Kumar Devarajan, New Delhi (IN)

Correspondence Address: LADAS & PARRY LLP, 26 WEST 61ST STREET, NEW YORK, NY, 10023, US

(73) Assignee: PANACEA BIOTEC LTD, New Delhi (IN)

(21) Appl. No.: 12/294,482

(22) PCT Filed: Mar. 19, 2007
(86) PCT No.: PCT/IN2007/000110

§ 371 Date: Sep. 25, 2008

(30) Foreign Application Priority Data
Mar. 27, 2006 (IN) 832/DEL/2006

(51) Int. Cl. A61K 38/16; A61K 31/7088; A61K 31/437; A61K 31/5513; A61K 31/5415; A61K 31/5517; A61K 31/554; A61K 31/496; A61P 25/18

(52) U.S. Cl. 514/12; 514/44 R; 514/285; 514/221; 514/226.2; 514/220; 514/211.13; 514/254.04

(57) ABSTRACT

Sustained release pharmaceutical composition comprising at least one poorly soluble active agent(s), at least one solubilizer, a release rate controlling polymer system consisting of an acid-soluble polymer and a pH-dependent polymer, and optionally other pharmaceutically acceptable excipients. The present invention also describes a process for preparation of such compositions and method of using such compositions. The sustained release compositions are useful in providing therapeutically effective levels of active agent(s) for extended periods of time.

### FIELD OF THE INVENTION

The present invention relates to novel sustained release pharmaceutical compositions comprising at least one poorly soluble active agent(s), at least one solubilizer(s), a release rate controlling polymer system, and optionally other pharmaceutically acceptable excipient(s). The present invention also describes process for preparation of such compositions and method of using such compositions. The sustained release compositions of the present invention are useful in providing therapeutically effective levels of active agent(s) for extended periods of time.

### BACKGROUND OF THE INVENTION

Sustained release pharmaceutical formulations provide a significant advantage over immediate release formulations to both clinicians and their patients. Sustained release dosage forms are administered to patients in much fewer daily doses than their immediate release counterparts and generally achieve improved therapeutic effect and efficiency in the fewer daily doses. For example, in a standard dosage regimen a 400 mg immediate release dosage form of an active ingredient (hereinafter “drug” or “medicament”) with a short half-life, may have to be administered to a patient two times within 24 hours to maintain adequate bioavailability of the drug to achieve therapeutic effect. This results in a series of two serum concentration profiles in the patient in which there is a rapid increase of drug followed by a similar rapid decrease. Such rapid increases and decreases provide a patient with a short window of appropriate blood concentration of the medicament for optimum therapy. Sustained release dosage forms generally control the rate of active drug absorption, so as to avoid excessive drug absorption while maintaining effective blood concentration of the drug to provide a patient with a consistent therapeutic effect over an extended duration of time.

Besides reducing the frequency of dosing and providing a more consistent therapeutic effect, sustained release dosage forms generally help reduce side effects caused by a drug. Because sustained release dosage forms deliver the drug in slow, incremental amounts versus the cyclic high and low concentrations of immediate release formulations, it is easier for a patient's body to digest the drug, thereby avoiding undesirable side-effects. For patients who self-administer therapies, sustained release dosage forms generally result in greater compliance due to the lower frequency of dosing, lower quantity of dosage units to be consumed, and reduced undesired side-effects.

Ziprasidone, 5-[2-[4-(1,2-benzisothiazol-3-yl)-1-piperazinyl]ethyl]-6-chloro-1,3-dihydro-2H-indol-2-one, is disclosed in U.S. Pat. No. 4,831,031. Ziprasidone is one of the newest atypical anti-psychotic agent, indicated for the treatment of schizophrenia. Ziprasidone is found to be efficacious in the improvement of positive symptoms in case of schizophrenic patients, which can be attributed to D2 receptor blockade within the limbic system. It is also known to possess moderate anti-depressant effects. Ziprasidone has shown to be effective in the management of acute mania in patients with bipolar disorders. The drug exhibits broad range of absorption in the gastrointestinal tract, extending from stomach upto the intestinal region, wherein presence of food doubles the absorption. Ziprasidone is well absorbed after oral administration, reaching peak plasma concentrations in 6 to 8 hours. The drug undergoes extensive metabolism due to aldehyde oxidase and cytochrome P450, having an oral bioavailability (60%).

Ziprasidone is generally administered orally (initial dose 20 mg BID) with food whereby the dose being increased upto 80 mg BID in cases where necessary. However, in cases of acute agitation in patients of schizophrenia, it may be given as the mesylate salt by intramuscular injection. It is generally available as oral capsules and intramuscular injectable preparation. Such frequent administration of conventional dosage form, gives an opportunity to develop an oral sustained release ziprasidone dosage form that provide efficacious blood levels of ziprasidone over a longer period of time than the IR formulation. Such a dosage form may increase patient compliance and maximize patient and physician acceptance, such as by reducing side effects. Such a dosage form may also provide a safety and tolerability profile as good as or better than the IR oral capsule regimen due to relatively lower blood levels of ziprasidone compared with the IR oral capsule at the same dose.

U.S. Pat. No. 6,150,366 describes a composition comprising crystalline ziprasidone freebase or crystalline ziprasidone hydrochloride particles having a mean particle size equal to or less than about 85 μm and a pharmaceutically acceptable diluent or carrier. PCT Publication No. WO2005020929 discloses a sustained release oral dosage form comprising a pharmaceutically effective amount of ziprasidone and a sustained release means for releasing at least a portion of said ziprasidone, wherein following administration to achieve steady state, said dosage form provides a steady state minimum blood ziprasidone concentration (Cₘᵢₙ) of at least 20 ng/ml, and a steady state maximum blood ziprasidone concentration (Cₘₐₓ) of less than 330 ng/ml. PCT Publication No. WO9741896 discloses a composition comprising a pharmaceutically acceptable salt of an aryl-heterocyclic compound, such as ziprasidone, in a cyclodextrin.

PCT Publication No. WO200579752 pertains to controlled release oral pharmaceutical composition comprising of a therapeutically effective amount of one or more pharmacologically active agents showing low bioavailability, one or more solubilizers, one or more biocompatible swelling agents, and a swelling enhancer. PCT Publication No. WO200541929 discloses a pharmaceutical composition comprising a therapeutically effective amount of a drug, a solubilizer, and a release modulator, wherein the release of the drug and solubilizer are synchronized.

PCT Publication No. WO200534920 describes a solid oral dosage form comprising a fibrate dissolved in a vehicle in order to ensure improved bioavailability of the active ingredient upon oral administration relative to known fibrate formulations, which is hydrophobic, hydrophilic or water-miscible. European patent No. EP249587 discloses a solid preparation with extended release of an active compound having a solubility less than 0.1 percent by weight in water, characterized in that it contains the active compound dissolved or dispersed in a semi-solid or liquid non-ionic solubilizer selected from esters and/or ethers of polyethyleneglycols and whereby the amount by weight of the solubilizer is at least equal to the amount by weight of the active compound and that the release is controlled by a hydrophilic gel system. US Publication No. 2005163858 discloses a formulation, comprising: an active agent, wherein the active agent is ziprasidone or a pharmaceutically acceptable salt thereof, wherein the active agent has a mean particle size greater than 85 micrometers; and a pharmaceutically acceptable carrier. PCT Publication No. WO2005123086 describes a dosage form comprising ziprasidone or a salt thereof in the form of particles having a mean size at least about 90 pm, and having a ziprasidone bioavailability equal to or greater than the bioavailability of a dosage form where ziprasidone or a salt thereof is present as particles having a mean size less than 85 pm.

Several attempts to provide dosage forms for delivery of active agent for extended periods of time have been described previously. However, there still exists a need to develop effective sustained release composition having reduced side effects which can provide sustained delivery of active agent, that is easier to manufacture, and involves a low formulation cost. Moreover, formulating ziprasidone into a sustained release dosage form presents a number of problems. While ziprasidone has relatively good solubility at gastric pH, it has relatively poor solubility at intestinal pH. The free base form of ziprasidone has a solubility of about 0.2 pg/ml at a pH of about 6.5. Such low solubility at intestinal pH inhibits absorption of ziprasidone in the intestines. In addition, if ziprasidone becomes supersaturated in an aqueous solution (that is, dissolved at a concentration that is greater than the equilibrium solubility of the drug at intestinal pH, such as occurs when moving from a low-pH gastric environment to a higher pH intestinal environment), it has a tendency to rapidly precipitate as the crystalline free base form of the drug, thus rapidly reducing the concentration of dissolved ziprasidone to the solubility of the free base crystalline (lowest energy form) of ziprasidone. The present invention overcomes the solubility issues of the ziprasidone in the GIT while the matrix dosage form moves from a low-pH gastric environment to a higher pH intestinal environment, there by providing a constant drug release over a period of time to achieve the therapeutic concentration of drug in the blood. The present invention provides such novel sustained release compositions.

### SUMMARY OF THE INVENTION

It is an objective of the present invention to provide novel sustained release pharmaceutical composition comprising at least one poorly soluble active agent(s), at least one solubilizer(s), a release rate controlling polymer system, and optionally other pharmaceutically acceptable excipients.

It is further an objective of the present invention to provide novel sustained release pharmaceutical composition comprising at least one poorly soluble active agent(s), at least one solubilizer(s), a release rate controlling polymer system which comprises of a combination of at least one acid soluble polymer(s) and at least one pH independent polymer(s), optionally with other pharmaceutically acceptable excipients.

It is also an objective of the present invention to provide novel sustained release pharmaceutical composition comprising at least one poorly soluble active agent(s), at least one solubilizer(s), a release rate controlling polymer system which comprises of a combination of at least one acid soluble polymer(s) and at least one pH independent polymer(s), optionally with other pharmaceutically acceptable excipients, wherein the said composition additionally comprises at least one hydration inhibitor(s).

It is also an objective of the present invention to provide novel sustained release pharmaceutical composition comprising at least one poorly soluble active agent(s), preferably antipsychotic drug(s), more preferably ziprasidone or its salts, polymorphs, solvates, hydrates, analogues, enantiomers, tautomeric forms, derivatives or mixtures thereof as active agent, either alone or in combination with other active agent(s); at least one solubilizer(s); a release rate controlling polymer system which comprises of a combination of at least one acid soluble polymer preferably that swells at about pH 5 and above and at least one pH independent polymer; at least one hydration inhibitor; optionally with other pharmaceutically acceptable excipients.

It is an objective of the present invention to provide process for preparation of such composition which comprises of the following steps:
i) mixing the active agent(s) with solubilizer(s), and release rate controlling polymer system,
ii) optionally adding one or more other excipient(s), and
iii) formulating the mixture into a suitable dosage form.

It is an objective of the present invention to provide process for preparation of such novel composition which comprises of the following steps:
i) mixing the active agent(s) with other excipients and hydration inhibitor(s) and granulating with a solubilizer(s),
ii) mixing the granules of step (i) with the release rate controlling system,
iii) optionally adding one or more other excipient(s), and
iv) formulating the mixture into a suitable dosage form.

It is yet another objective of the present invention to provide a method of using such composition which comprises administering to a subject in need thereof an effective amount of the composition.

The novel compositions of the present invention provide therapeutic concentrations of active agent(s) for extended periods of time.

### DETAILED DESCRIPTION OF THE INVENTION

The present invention provides novel sustained release pharmaceutical composition comprising at least one poorly soluble active agent(s), at least one solubilizer(s), a release rate controlling polymer system, and optionally other pharmaceutically acceptable excipients. In an embodiment, the active agent(s) used in the present invention is preferably antipsychotic agent(s), more preferably ziprasidone or its salts, polymorphs, solvates, hydrates, analogues, enantiomers, tautomeric forms, derivatives or mixtures thereof.

In an embodiment, the present invention provides novel sustained release pharmaceutical composition comprising at least one poorly soluble active agent(s), at least one solubilizer(s), a release rate controlling polymer system which comprises of a combination of at least one acid soluble polymer(s) and at least one pH independent polymer(s), additionally comprises at least one hydration inhibitor(s), optionally with other pharmaceutically acceptable excipients.

In an embodiment, the present invention provides novel sustained release pharmaceutical composition comprising at least one poorly soluble active agent(s), at least one solubilizer(s), a release rate controlling polymer system which comprises of a combination of at least one acid soluble polymer(s) and at least one pH independent polymer(s), optionally with other pharmaceutically acceptable excipients, wherein the said composition additionally comprises at least one hydration inhibitor(s).

In another embodiment of the present invention, the solubilizer is present in an amount not less than about 2.5% preferably not less than about 5% by weight of the composition. In another embodiment, the release rate controlling polymer system comprises of a combination of at least one acid soluble polymer(s) preferably that swells at about pH 5 and above and at least one pH independent polymer(s), wherein acid soluble polymer is present in an amount not less than about 5% by weight of the composition and pH independent polymer is present in an amount not less than about 2.5% by weight of the composition. In another embodiment, the acid soluble polymer preferably swells at about pH 5 and above. In another embodiment, the hydration inhibitor is present in an amount not less than about 5% by weight of the composition.

In a preferred embodiment, the present invention provides novel sustained release pharmaceutical composition wherein the said system releases the active agent(s) predominantly by erosion mechanism without any substantial deformation of shape and provides therapeutic concentrations of active agent(s) for extended periods of time.

In an embodiment, the novel controlled release pharmaceutical compositions of the present invention are intended to reduce the adverse effects or side effects of the active agent(s) by controlling the peak plasma concentration (Cₘₐₓ) such that the concentration of the active agent(s) is substantially below the toxic levels at any point of time. Also the steady state concentrations of the active agent(s) do not exhibit substantial fluctuations. The reduced incidence of these neurological side effects is thus intended to improve patient compliance with the therapy.

In an embodiment, the novel compositions of the present invention release the active agent preferably for a period of about 8-24 hours, optionally having an initial lag time wherein only 0 to about 15% of active agent is released, followed by a sustained release of active agent. The present system preferably used for controlling release rate in the present invention comprises of at least one solubilizer and a release rate controlling polymer system. The said system is unique because presence of solubilizer contributes towards the solubility of the drug in aqueous fluids and a release rate controlling polymer system which comprises of a combination of at least one acid soluble polymer preferably that swells at about pH 5 and above and at least one pH independent polymer, it provides the desired release profile of the active agent, wherein acid soluble polymer regulates the releasing rate in acidic environment of GIT and pH independent polymer regulates the releasing rate in entire GIT by maintaining the intactness of dosage form and also providing the release in intestine although active agent has negligible solubility in higher pH. Additionally, inclusion of at least one hydration inhibitor(s) intends to maintain the tablet core integrity for longer duration of time. Furthermore, the dosage form compositions of the present invention do not require the incorporation of any de-agglomerating excipient such as silicon dioxide to control release of the active agent from the compositions.

In an embodiment of present invention, sustained release systems can be formulated in the form of mucoadhesive matrix type dosage forms wherein the drug is dissolved and/or dispersed in the polymer matrix system. The dosage form binds to the gastro-intestinal tract in a pH range of about 1.2 to about 6.8. Preferably in mucoadhesive type dosage form the release of drug from the dosage form is by diffusion through hydrogel formation due to swelling of the polymer component(s) of the system and/or controlled erosion of the system. The pharmaceutical composition of the present invention comprises at least one acid soluble polymer(s) that preferably swells in an aqueous environment, and which can also act as a mucoadhesive polymer.

The active agent of the present invention is selected from but not limited to a group comprising active agent(s) such as cardiovascular drug, respiratory drug, sympathomimetic drug, cholinomimetic drug, adrenergic agonist, adrenergic antagonist, analgesic/antipyretic, anesthetic, antiasthamatic, antibiotic, antidepressant, antidiabetic, antifungal agent, antihypertensive agent, anti-inflammatory, antineoplastic, antianxiety agent, immunosuppressive agent, antimigraine agent, sedative/hypnotic, antianginal agent, antipsychotic agent, antimanic agent, antiarrhythmic, antiarthritic agent, antigout agent, anticoagulant, thrombolytic agent, antifibrinolytic agent, hemorheologic agent, antiplatelet agent, anticonvulsant, antiparkinson agent, antihistaminic/antipruritic, agent useful for calcium regulation, antibacterial agent, antiviral agent, antimicrobial, anti-infective, bronchodialator, hormone, hypoglycemic agent, hypolipidemic agent, protein, nucleic acid, agent useful for erythropoiesis stimulation, antiulcer/antireflux agent, antinauseant/antiemetic, oil-soluble vitamin, mitotane, visadine, halonitrosourea, anthrocycline or ellipticine and their pharmaceutically acceptable salts, esters, amides, polymorphs, solvates, hydrates, analogues, enantiomers, tautomeric forms or mixtures thereof, used either alone or in combination thereof.

Preferably the active agent of the present invention is an antipsychotic agent selected from but not limited to a group comprising emonaprode, diazepam, nitrazepam, flunitrazepam, lorazepam, prazepam, fluidiazepam, clonazepam, chlorpromazine hydrochloride, reserpine, clofluperol, trifluperidol, haloperidol, moperone, bromperidom, aripiprazole, sertindole, amisulpiride, asenapine, paloperidone or blonanserine, flupenthixol, fluphenazin, perphenazin, pimozide, chlorpromazine, tioridazine, melperone, zuclpentixol, etizolam, risperidone, olanzapine, clozapine, mipiprazole, quetiapine, ziprasidone or pharmaceutically acceptable salts, hydrates, polymorphs, esters, and derivatives thereof used either alone or in combination thereof.

In an embodiment, the active agent(s) used in the present invention is preferably antipsychotic agent(s), more preferably ziprasidone or its salts, polymorphs, solvates, hydrates, analogues, enantiomers, tautomeric forms, derivatives or mixtures thereof. In an embodiment of the present invention, the active agent is ziprasidone hydrochloride, which is substantially amorphous, semicrystalline or crystalline in nature, or mixtures thereof. In another embodiment of the present invention, the active agent is ziprasidone hydrochloride, which is in anhydrous or hydrated form or mixtures thereof. The hydrated form may be one or more of hemihydrate, monohydrate, dihydrate, trihydrate and tetrahydrate.

In another embodiment, the mean particle size of the active agent such as ziprasidone hydrochloride ranges from about 0.2 micron to about 2000 microns, preferably about 1 micron to about 1000 microns. In another embodiment of the present invention, the active agent is in the form of crystalline ziprasidone hydrochloride particles, having a mean particle size of less than about 5 microns. In yet another embodiment of the present invention, the active agent is in the form of crystalline ziprasidone hydrochloride particles, having a mean particle size of more than about 220 microns. In still further embodiment, the composition of the present invention comprises the active agent ziprasidone hydrochloride in a substantially amorphous form. The active agent can be made into an amorphous form by preparing it as a solid dispersion using hot melt, wet granulation, spray drying or lyophilizing technique, or combination of such techniques, or any other technique known to the art.

In an embodiment of the present invention, the solubilizer is selected from but not limited to a group comprising hydrophilic surfactants or lipophilic surfactants or mixtures thereof. The surfactants may be anionic, nonionic, cationic, or zwitterionic surfactants, or mixtures thereof.

The hydrophilic non-ionic surfactants may be selected from the group comprising but not limited to polyethylene glycol sorbitan fatty acid esters and hydrophilic transesterification products of a polyol with at least one member of the group consisting of triglycerides, vegetable oils, and hydrogenated vegetable oils preferably glycerol, ethylene glycol, polyethylene glycol, sorbitol, propylene glycol, pentaerythritol, or a saccharide, d-tocopheryl polyethylene glycol 1000 succinate.

The ionic surfactants may be selected from the group comprising but not limited to alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and diglycerides; succinylated mono- and di-glycerides; citric acid esters of mono- and diglycerides; and mixtures thereof.

The lipophilic surfactants may be selected from the group comprising but not limited to fatty alcohols; glycerol fatty acid esters; acetylated glycerol fatty acid esters; lower alcohol fatty acid esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; PEG sorbitan fatty acid esters, PEG glycerol fatty acid esters, polyglycerized fatty acid, polyoxyethylene-polyoxypropylene block copolymers, sorbitan fatty acid esters; and mixtures thereof.

Preferably the solubilizer may be selected from PEG glyceryl stearate (Capmul®), PEG-40 hydrogenated castor oil (Cremophor®), PEG 6 corn oil (Labrafl®), lauryl macrogol—32 glyceride (Gelucire® 44/14), stearoyl macrogol glyceride (Gelucire® 50/13), polyglyceryl-10 dioleate (Caprol®), propylene glycol oleate (Lutrol®), mono Propylene glycol dioctanoate (Captex®), Propylene glycol caprylate/caprate (Labrafac®), Glyceryl monooleate (Peceole®), Glycerol monolinoleate (Maisine®), PEG sorbitan monolaurate (Tween®), PEG lauryl ether (Brij®), Sucrose distearate (Sucroester®), polyoxyethylene-polyoxypropylene block copolymer (Lutrol®)), polyethylene glycol hydroxystearate, (Solutol®), Sodium lauryl sulphate, Sodium dodecyl sulphate, Dioctyl suphosuccinate, L-hydroxypropyl cellulose, hydroxyethylcellulose, hydroxy propylcellulose, Propylene glycol alginate, sodium taurocholate, sodium glycocholate, sodium deoxycholate, betains, polyethylene glycol (Carbowax®), d-tocopheryl polyethylene glycol 1000 succinate (Vitamin E TPGS) and mixtures thereof. A more preferred solubilizer may be selected from PEG hydrogenated castor oil (Cremophor®), lauryl macrogol glyceride (Gelucire® 44/14), stearoyl macrogol glyceride (Gelucire® 50/13), PEG sorbitan monolaurate (Tween®), PEG lauryl ether (Brij®), polyoxyethylenepolyoxypropylene block copolymer (Lutrol®), Sodium lauryl sulphate, Sodium dodecyl sulphate, polyethylene glycol (Carbowax®) and mixtures thereof. Preferably the solubilizer used is stearoyl macrogol glyceride (Gelucire® 50/13). Preferably the stearoyl macrogol glyceride (Gelucire® 50/13) is present in an amount not less than 5% w/w of the composition, most preferably about 7-20% w/w of the composition.

In an embodiment of the present invention, the release rate controlling polymer system comprises of a combination of at least one acid soluble polymer(s) and at least one pH independent polymer(s). In further embodiment of the present invention, acid soluble polymer is preferably that swells at about pH 5 and above.

In an embodiment of the present invention, the acid soluble polymer is selected from but not limited to a group comprising polyalkylene oxides such as polyethylene oxide; cellulosic polymers such as methyl cellulose, hydroxypropyl cellulose, hydroxypropyl methylcellulose and hydroxyethyl cellulose; maleic anhydride polymers; poly(acrylamides); polyols; polyvinylamines; starch and starch-based polymers; polyurethane hydrogels; chitosan and its derivatives; polysaccharide gums; polyvinyl alcohol copolymers and the like or mixtures thereof.

Preferably the acid soluble polymer that swells at about pH 5 and above is chitosan. Preferably the chitosan is present in an amount not less than about 10% w/w of the composition, most preferably about 20 to 50% w/w of the composition. In another embodiment, preferably the acid soluble polymer is a derivative of chitosan such as acetylated chitosan.

In an embodiment of the present invention, the pH independent polymer is selected from but not limited to a group comprising alkyl celluloses such as methyl cellulose, hydroxyalkyl alkyl celluloses such as hydroxypropyl methyl cellulose (HPMC, Methocel®), hydroxy alkyl celluloses such as hydroxypropyl cellulose (HPC, Klucel®) and hydroxy ethyl cellulose (HEC, Natrosol®), polyethylene glycols (PEG®, Lutrol®), copolymers of ethylene oxide with propylene oxide (Poloxamer®), gelatin, polyvinylpyrrolidones (PVP, Kollidon®), vinylpyrrolidones, vinyl acetates, polyvinylimidazoles, polyvinylpyridine N-oxides, copolymers of vinylpyrrolidone with long-chained alpha.-olefins, copolymers of vinylpyrrolidone with vinylimidazole, poly(vinylpyrrolidone/dimethylaminoethyl methacrylates), copolymers of vinylpyrrolidone/dimethylaminopropyl methacrylamides, copolymers of vinylpyrrolidone/dimethylaminopropyl acrylamides, quaternised copolymers of vinylpyrrolidones and dimethylaminoethyl methacrylates, terpolymers of vinylcaprolactam/vinylpyrrolidone/dimethylaminoethyl methacrylates, copolymers of vinylpyrrolidone and methacrylamidopropyl-trimethylammonium chloride, terpolymers of caprolactam/vinylpyrrolidone/dimethylaminoethyl methacrylates, copolymers of styrene and acrylic acid, polycarboxylic acids, polyacrylamides, polyvinyl alcohols (PVA, Mowiol®), hydrolysed polyvinyl acetate, copolymers of ethyl acrylate with methacrylate and methacrylic acid, copolymers of maleic acid with unsaturated hydrocarbons and mixed polymerisation products of the said polymers, polysaccharide gums, both natural and modified (semi-synthetic), including but not limited toxanthan gum, veegum, agar, guar gum, locust bean gum, gum arabic, okra gum, alginic acid, other alginates (e.g. sodium alginate HVCR, propyleneglycol alginate), benitonite, arabinoglactin, pectin, tragacanth, scleroglucan, dextran, amylose, amylopectin, dextrin, and the like or mixtures thereof.

Preferably the pH independent polymer is hydroxyalkyl alkyl celluloses, more preferably is hydroxypropyl methylcellulose. Preferably the hydroxypropyl methylcellulose is present in an amount not less than about 2.5% w/w of the composition, most preferably about 10% to about 30% w/w of the composition. In a preferred embodiment of the present invention, the ratio of the acid soluble polymer and the pH independent polymer is about 1:50 to about 50:1, preferably about 1:30 to about 30:1 by weight of the composition.

In a preferred embodiment, novel controlled release pharmaceutical composition additionally comprises at least one hydration inhibitor(s), preferably a combination of at least two hydration inhibitors. In an embodiment of the present invention, hydration inhibitor is selected from but not limited to a group comprising stearic acid, glyceryl monostearate, glyceryl behenate (Compritol® 888 ATO), glyceryl monooleate, glyceryl palmitostearate, microcrystalline wax, stearyl alcohol, cetyl alcohol, cetostearyl alcohol, hydrogenated castor oil, tristearin, waxes, polyvinyl acetates, polyethylenes, polypropylenes, polyamides, ethylene glycol polyterephthalate, polyvinyl chlorides, polyformaldehyde chlorides, polycarbonates, ethylene copolymers, polyethers, polyurethanes, polyacrylonitriles, shellac, rosin, dibasic calcium phosphate or mixtures thereof. In a preferred embodiment, novel controlled release pharmaceutical composition comprises at least one hydration inhibitor(s) in an amount not less than about 5% by weight of the composition. Preferably the hydration inhibitor(s) is present in an amount of about 10% to about 20% by weight of the composition. In another preferred embodiment, the composition of the present invention comprises a combination of hydration inhibitors. The combination of hydration inhibitors comprise of glyceryl behenate and dibasic calcium phosphate. In a further preferred embodiment of the present invention, the ratio of glyceryl behenate to dibasic calcium phosphate is about 1:10 to about 10:1, preferably about 1:5 to about 5:1 by weight of the composition.

In an embodiment, the composition of the present invention additionally comprises excipients selected from but not limited to a group comprising diluent and a solvent. In an embodiment of the present invention, the diluent is selected from but not limited to a group comprising such as microcrystalline cellulose, lactose, starch, dibasic calcium phosphate, saccharides, and/or mixtures of the foregoing. Examples of diluents include microcrystalline celluloses (Avicel®); lactose such as lactose monohydrate, lactose anhydrous (Pharmatose®), including anhydrous, monohydrate and spray dried forms; dibasic calcium phosphate (Emcompress®); mannitol (Pearlitol®); starch; sorbitol; sucrose; glucose; cyclodextrins; or the like or mixtures thereof. In the present invention, the solvent used is selected from but not limited to a group comprising alcohols such as methanol, ethanol, propanol, isopropyl alcohol, butanol, monomethoxyethanol, ethylene glycol monomethylether and the like; ethers such as diethyl ether, dibutyl ether, diisobutyl ether, dioxane, tetrahydrofuran, ethylene glycol and the like; aliphatic hydrocarbons such as n-hexane, cyclohexane and n-heptane; aromatic hydrocarbons such as benzene, toluene and xylene; nitriles such as acetonitrile and the like; organic acids such as acetic acid, propionic acid and the like; esters such as ethyl acetate; aliphatic halogenated hydrocarbons such as dichloromethane, dichloroethane, chloroform and the like; ketones such as acetone, methyl ketone and the like; amides such as dimethylformamide, dimethyl acetamide and the like; or mixtures thereof. Among the solvents, the one having a low boiling point such as ketones e.g. acetone and alcohols e.g. ethanol is preferable. More preferably the solvent used is dichloromethane and is in a quantity sufficient to dissolve or disperse the solubilizer and/or the active agent(s).

The pharmaceutically acceptable excipients of the present invention are selected from but not limited to a group comprising diluents, disintegrants, binders, mucoadhesive agents, fillers, bulking agents, anti-adherants, anti-oxidants, buffering agents, complexing agents, carriers, colorants, flavoring agents, coating agents, plasticizers, organic solvents, stabilizers, preservatives, lubricants, solubilizers, glidants, chelating agents, and the like known to the art used either alone or in combination thereof. It will be appreciated that certain excipients used in the present composition can serve more than one purpose.

Suitable mucoadhesive agents include for example thiolated polymers (thiomers), glycoproteins, synthetic polymers such as poly(acrylic acid) (PAA), hydroxypropyl methylcellulose and poly(methylacrylate) derivatives, naturally occurring polymers such as hyaluronic acid and chitosans, certain carbohydrates, plant lectins, bacterial adhesins, methylcellulose, sodium carboxymethyl cellulose, carbopol and the like. Suitable binders include for example starch, polyvinylpyrrolidone, povidone, hydroxypropyl methylcellulose, pregelatinised starch, hydroxypropylcellulose or mixtures thereof. Suitable lubricants are selected from but not limited to a group comprising colloidal silicon dioxide such as Aerosil® 200; talc; stearic acid, magnesium stearate, calcium stearate, sodium stearyl fumarate, hydrogenated vegetable oil and the like or mixtures thereof. Suitable disintegrants include for example crosslinked polyvinyl pyrrolidone, corn starch, potato starch, maize starch and modified starches, croscarmellose sodium, sodium starch glycollate, carboxymethyl cellulose calcium, or mixtures thereof. Suitable carrier is selected from but not limited to a group comprising crospovidone, cross-linked polymeric cyclodextrin, dextran, cellulose, alginates, silica gel, titanium dioxide, aluminum oxides; cellulose derivatives such as microcrystalline cellulose, hydroxypropyl cellulose, hydroxypropyl methylcellulose; starches such as cross-linked sodium carboxymethyl starch, maize, rice, corn and potato starch, polyethylene glycols; sugars, saccharide such as lactose and dextrose; sugar alcohols, such as sorbitol or mannitol; non-pareil seed such as Microcrystalline Cellulose Spherical Seed Core (Celphere®); croscarmellose sodium (Ac-Di-sol®), sodium starch glycolate, polyvinyl alcohol, ascorbic acid, carbopols, polyethylene oxide, mixtures of mono-, di-, and triglycerides with polyethylene glycol (PEG) esters of fatty acids such as Gelucires, and the like or mixtures thereof. Suitable complexing agents include for example cyclodextrin, preferably with a beta-cyclodextrin, more preferably with Hydroxypropyl beta cyclodextrin and the like.

In another embodiment, the composition of the present invention is formulated as a layered tablet comprising at least one immediate release (IR) layer and one sustained release (SR) layer. The IR layer is intended to provide fast release of the active agent(s) and the SR layer is intended to provide a sustained release of the active agent. In a further embodiment, the composition of the present invention comprises of at least two fractions wherein one fraction comprises the active agent(s) and optionally other pharmaceutically acceptable excipients in such quantities so as to provide an immediate release of the active agent(s) and the other fraction comprises the active agent(s), at least one solubilizer(s), a release rate controlling polymer system, and optionally other pharmaceutically acceptable excipients in such quantities so as to provide a sustained release of the active agent(s).

In a further embodiment, the compositions of the present invention comprising pharmaceutically active agent(s) were subjected to in vitro dissolution study in a dissolution media having a pH ranging from 1 to 9, preferably having a pH less than about 4-7. About 0-40% of the active agent(s) was released within about 2-4 hours and greater than about 40% of the active agent(s) was released after about 8 hours of test. In a still further embodiment, the compositions of the present invention are studied in healthy human volunteers. The time taken to reach the peak plasma concentration (Cₘₐₓ) by the compositions of the present invention is in the range of about 0.5-12 hours (Tₘₐₓ), preferably in the range of about 1-10 hours. However, it might be emphasized that the selection of the in vitro dissolution study media, the parameters and apparatus is made in such a manner so as to provide a scientific rationale to the intended study and/or a logical correlation to the in vivo data as understood by a person skilled in art, and any modifications in such study either in vitro or in vivo is within the purview of the present invention.

In an embodiment of the present invention is provided a process for the preparation of such novel composition which comprises of the following steps:
i) mixing the active agent(s) with solubilizer(s), and release rate controlling polymer system,
ii) optionally adding one or more other excipient(s), and
iii) formulating the mixture into a suitable dosage form.

In another embodiment of the present invention is provided a process for the preparation of such novel composition which comprises of the following steps:
i) mixing the active agent(s) with other excipients and hydration inhibitor(s) and granulating with a solubilizer(s),
ii) mixing the granules of step (i) with the release rate controlling system,
iii) optionally adding one or more other excipient(s), and
iv) formulating the mixture into a suitable dosage form.

In another embodiment of the present invention is provided a process for the preparation of such novel composition which comprises of the following steps:
i) mixing the active agent(s) with a portion of release rate controlling polymer system and hydration inhibitor(s) and granulating with a solubilizer(s),
ii) mixing the granules of step (i) with remaining portion of release rate controlling polymer system,
iii) optionally adding one or more other excipient(s), and
iv) formulating the mixture into a suitable dosage form.

In yet another embodiment of the present invention is provided a process for the preparation of such novel composition which comprises of the following steps:
i) mixing the active agent(s) with other excipients,
ii) mixing the material of step (i) with the release rate controlling system,
iii) mixing the blend of step (ii) with hydration inhibitor(s) and other excipient(s),
iv) granulating the material of step (iii) with the solubilizer(s), and
v) formulating the mixture into a suitable dosage form.

In an embodiment, the amorphous form of the active agent(s) useful in the present invention is prepared by employing the solid dispersion technique wherein the solubilizer is melted and the active agent(s) is dissolved/dispersed in the solubilizer, optionally followed by incorporation of an adsorbent material leading to a free flowing powder which is then further processed with other excipient(s) into suitable dosage form. In another embodiment, the amorphous form is prepared by mixing the active agent(s) along with the solubilizer in an aqueous or non-aqueous solution and then spray dried or lyophilized by using techniques known to the art to obtain dry powder which is then further processed with other excipient(s) into suitable dosage form.

In a further embodiment, the present composition can be formulated by spray drying or lyophilizing the active agent with a suitable solubilizer or low viscosity pH independent polymers to get free flowing powder. In an embodiment, active agent is preferably formulated as an aqueous or non-aqueous solution with said solubilizer or pH independent polymers and then spray dried or lyophilized using techniques known to the art to obtain dry powder which is then further processed with other excipient(s) into suitable dosage form.

In a further embodiment, the present composition can be formulated by spray drying or lyophilizing the active agent with complexing agent to make complex. In an embodiment, active agent is preferably formulated as an aqueous or non-aqueous solution with said complexing agent and then spray dried or lyophilized using techniques known to the art to obtain dry powder which is then further processed with other excipient(s) into suitable dosage form.

In a further embodiment, the present composition can be formulated by spraying the active agent with binder on to an inert carrier by using fluid bed coater, mixing the granules with the acid soluble polymer(s) and the pH independent polymer(s), optionally adding one or more other excipient(s), and formulating the mixture into a suitable dosage form.

In a further embodiment, the composition of the present invention is preferably in the form of solid dosage forms such as tablets, capsules, pellets or the like, more preferably as tablets. The tablets can be prepared by either wet granulation, direct compression, or by dry compression (slugging). In a preferred embodiment of the present invention, the oral composition is prepared by wet granulation. The granulation technique is either aqueous or non-aqueous. The non-aqueous solvent used is selected from a group comprising acetone, ethanol, isopropyl alcohol or methylene chloride. In an embodiment, the compositions of the present invention are in the form of compressed tablets, moulded tablets, mini-tablets, capsules, pellets, granules and products prepared by extrusion or film cast technique, and the like. The tablets may be optionally coated with a nonfunctional coating to form a nonfunctional layer. The tablets/minitablets may be optionally filed into capsules.

In yet another embodiment of the present invention is provided a method of using such novel sustained release compositions which comprises administering to a subject in need thereof an effective amount of the composition. The compositions comprising antipsychotic agent(s) as the active agent are useful for the management of psychosis and psychotic symptoms for example, schizophrenia, an obsessive compulsive disorder, depression, a bipolar disorder, or Tourette's syndrome. The psychotic symptoms can include delusions, hallucinations, disorganized speech, grossly disorganized or catatonic behavior, and the like. The examples given below serve to illustrate embodiments of the present invention. However they do not intend to limit the scope of present invention.

### EXAMPLES

### Example-1

| | | |
|---|---|---|
| S. No. | Ingredient | mg/tablet |

Core composition

| | | |
|---|---|---|
| 1. | Ziprasidone hydrochloride | 46.39 |
| 2. | Stearoyl macrogol glyceride | 45.00 |
| | (Gelucire ® 50/13) | |
| 3. | Dibasic calcium phosphate | 55.00 |
| 4. | Chitosan | 80.00 |
| 5. | Hydroxypropyl methylcellulose | 88.00 |
| | (Hypromellose ® 2208) | |
| 6. | Polyvinylpyrrolidone (PVP K ®-90) | 30.00 |
| 7. | Glyceryl behenate (Compritol ® 888) | 48.00 |
| 8. | Dichloromethane (DCM) | q.s. (lost in processing) |
| 9. | Magnesium stearate | 8.00 |

Coating composition

| | | |
|---|---|---|
| 10. | Opadry ® orange (in water) | q.s. |

### Procedure:

i) Ziprasidone hydrochloride and Dibasic calcium phosphate were mixed together.
ii) Chitosan and Hydroxypropyl methylcellulose were mixed together separately.
iii) Blend of step (i) was mixed with blend of step (ii).
iv) Polyvinylpyrrolidone and Glyceryl behenate were added to the mixture of step (iii) and was sifted from # 40 sieve.
v) Stearoyl macrogol glyceride was dissolved in Dichloromethane.
vi) Blend of step (iv) was granulated with the solution of step (v) and was passed through # 30 sieve.
vii) The granules of step (vi) was dried and mixed with half quantity of Magnesium stearate.
viii) The blend of step (vii) was compacted and passed through the # 40 sieve.
ix) The granules of step (viii) were mixed with remaining quantity of Magnesium stearate and compressed into tablets.
x) The tablets of step (ix) were coated with the Opadry® orange (in water) and dried.

### Example-2

| | | |
|---|---|---|
| S. No. | Ingredient | mg/tablet |

Core composition

| | | |
|---|---|---|
| 1. | Ziprasidone hydrochloride | 46.39 |
| 2. | Stearoyl macrogol glyceride | 45.00 |
| | (Gelucire ® 50/13) | |
| 3. | Glyceryl behenate (Compritol ® 888) | 50.00 |
| 4. | Chitosan | 80.00 |
| 5. | Hydroxypropyl methylcellulose | 88.00 |
| | (Hypromellose ® 2208) | |
| 6. | Polyvinylpyrrolidone (PVP K ®-90) | 30.00 |
| 7. | Dichloromethane (DCM) | q.s. (lost in processing) |
| 8. | Magnesium stearate | 8.00 |

Coating composition

| | | |
|---|---|---|
| 9. | Opadry ® orange (in water) | q.s. |

### Procedure:

i) Chitosan and Hydroxypropyl methylcellulose were mixed together.
ii) Ziprasidone hydrochloride was mixed with blend of step (i).
iii) Polyvinylpyrrolidone and Glyceryl behenate were added to the mixture of step (ii) and was sifted from # 40 sieve.
iv) Stearoyl macrogol glyceride was dissolved in Dichloromethane.
v) Blend of step (iii) was granulated with the solution of step (iv).
vi) The material of step (v) was passed through # 30 sieve.
vii) The granules of step (vi) were dried and mixed with half quantity of Magnesium stearate.
viii) The blend of step (vii) was compacted and passed through the # 40 sieve.
ix) The granules of step (viii) were mixed with remaining quantity of Magnesium stearate and compressed into tablets.
x) The tablets of step (ix) were coated with the Opadry® orange (in water) and dried.

### Example-3

### A. Preparation of Sustained Release Fraction

| S. No. | Ingredients | mg/tablet |
|---|---|---|
| 1. | Ziprasidone hydrochloride | 37.11 |
| 2. | Stearoyl macrogol glyceride | 37.00 |
| 3. | Dibasic calcium phosphate | 40.00 |
| 4. | Chitosan | 80.00 |
| 5. | Hydroxypropyl methylcellulose | 88.00 |
| 6. | Polyvinylpyrrolidone | 30.00 |
| 7. | Dichloromethane (DCM) | q.s. (lost in processing) |
| 8. | Magnesium stearate | 8.00 |

### Procedure:

i) Chitosan and Hydroxypropyl methylcellulose were mixed together.
ii) Ziprasidone hydrochloride was mixed with blend of step (i).
iii) Polyvinylpyrrolidone and Dibasic calcium phosphate were added to the mixture of step (ii) and was sifted from # 40 sieve.
iv) Stearoyl macrogol glyceride was dissolved in Dichloromethane.
v) Blend of step (iii) was granulated with the solution of step (iv).
vi) The material of step (v) was passed through # 30 sieve.
vii) The granules of step (vi) were dried and mixed with Magnesium stearate.

### B. Preparation of Immediate Release Fraction

| S. No. | Ingredients | mg/tablet |
|---|---|---|
| 1. | Ziprasidone hydrochloride | 9.28 |
| 2. | Microcrystalline cellulose | 120.00 |
| 3. | Low substituted hydroxypropyl cellulose | 6.50 |
| 4. | Magnesium stearate | 1.22 |

### Procedure:

i) Ziprasidone hydrochloride, Microcrystalline cellulose and Low substituted hydroxypropyl cellulose were mixed together.
ii) Magnesium stearate was sifted through sieve #40 and added to the material of step (i) followed by mixing.

### C. Tablet

i) The blend obtained in step (vii) of A and the blend of step (ii) of B was compressed into a tablet.

### Example-4

| S. No. | Ingredient | mg/tablet |
|---|---|---|
| 1. | Ziprasidone hydrochloride | 46.39 |
| 2. | Stearoyl macrogol glyceride | 45.00 |
| | (Gelucire ® 50/13) | |
| 3. | Anhydrous lactose | 12.00 |
| 4. | Chitosan | 187.11 |
| 5. | Hydroxypropyl methylcellulose | 71.50 |
| | (Hypromellose ® 2208) | |
| 6. | Polyvinylpyrrolidone (PVP K ®-90) | 30.00 |
| 7. | Dichloromethane (DCM) | q.s. (lost in processing) |
| 8. | Magnesium stearate | 8.00 |

### Procedure:

i) Ziprasidone hydrochloride and Anhydrous lactose were mixed together.
ii) Chitosan and Hydroxypropyl methylcellulose were mixed together separately.
iii) Blend of step (i) was mixed with blend of step (ii) and homogeneous mixture was formed.
iv) Polyvinylpyrrolidone was added to the homogeneous mixture of step (iii) and was sifted from # 40 sieve.
v) Stearoyl macrogol glyceride was dissolved in Dichloromethane.
vi) Blend of step (iv) was granulated with the solution of step (v) and was passed through # 30 sieve.
vii) The granules of step (vi) was dried and mixed with half quantity of Magnesium stearate.
viii) The blend of step (vii) was compacted and passed through the # 30 sieve.
ix) The granules of step (viii) were mixed with remaining quantity of Magnesium stearate and compressed into tablets.

### Example-5

| | | |
|---|---|---|
| S. No. | Ingredient | mg/tablet |

Core composition

| | | |
|---|---|---|
| 1. | Ziprasidone hydrochloride | 46.39 |
| 2. | Stearoyl macrogol glyceride | 45.00 |
| | (Gelucire ® 50/13) | |
| 3. | Anhydrous lactose | 12.00 |
| 4. | Chitosan | 187.11 |
| 5. | Hydroxypropyl methylcellulose | 71.50 |
| | (Hypromellose ® 2208) | |
| 6. | Polyvinylpyrrolidone (PVP K ®-90) | 30.00 |
| 7. | Dichloromethane (DCM) | q.s. (lost in processing) |
| 8. | Magnesium stearate | 8.00 |

Coating composition

| | | |
|---|---|---|
| 9. | Opadry ® yellow (in water) | q.s. |

### Procedure:

i) Ziprasidone hydrochloride and Anhydrous lactose were mixed together.
ii) Chitosan and Hydroxypropyl methylcellulose were mixed together separately.
iii) Blend of step (i) was mixed with blend of step (ii) and homogeneous mixture was formed.
iv) Polyvinylpyrrolidone was added to the homogeneous mixture of step (iii) and was sifted from # 40 sieve.
v) Stearoyl macrogol glyceride was dissolved in Dichloromethane.
vi) Blend of step (iv) was granulated with the solution of step (v) and was passed through # 30 sieve.
vii) The granules of step (vi) was dried and mixed with half quantity of Magnesium stearate.
viii) The blend of step (vii) was compacted and passed through the # 30 sieve.
ix) The mixture of step (viii) was mixed with remaining quantity of Magnesium stearate and compressed into tablets.
x) The tablets of step (ix) were coated with the Opadry® yellow (in water) and dried.

### Example-6

| S. No. | Ingredient | mg/tablet |
|---|---|---|
| 1. | Bumetanide | 2.00 |
| 2. | Lauryl macrogol glyceride | 13.50 |
| | (Gelucire ® 44/14) | |
| 3. | Microcrystalline cellulose | 12.00 |
| 4. | Chitosan | 42.00 |
| 5. | Hydroxyethyl cellulose | 21.00 |
| 6. | Polyvinylpyrrolidone (PVP K ®-90) | 7.50 |
| 7. | Isopropyl alcohol | q.s. (lost in processing) |
| 8. | Magnesium stearate | 2.00 |

### Procedure:

i) Bumetanide and Microcrystalline cellulose were mixed together.
ii) Chitosan and Hydroxyethyl cellulose were mixed together separately.
iii) Blend of step (i) was mixed with blend of step (ii) and homogeneous mixture was formed.
iv) Polyvinylpyrrolidone was added to the homogeneous mixture of step (iii) and was sifted from # 40 sieve.
v) Lauryl macrogol glyceride was dissolved in Isopropyl alcohol.
vi) Blend of step (iv) was granulated with the solution of step (v) and was passed through # 30 sieve.
vii) The granules of step (vi) was dried and mixed with half quantity of Magnesium stearate.
viii) The blend of step (vii) was compacted and passed through the # 30 sieve.
ix) The mixture of step (viii) was mixed with remaining quantity of Magnesium stearate and compressed into tablets.

### Example-7

| S. No. | Ingredient | mg/capsule |
|---|---|---|
| 1. | Quetiapine fumarate | 38.44 |
| 2. | Propylene glycol caprylate/caprate | 42.00 |
| | (Labrafac ®) | |
| 3. | Microcrystalline cellulose | 10.00 |
| 4. | Polyethylene oxide | 150.00 |
| 5. | Hydroxyethyl cellulose | 65.56 |
| 6. | Isopropyl alcohol | q.s. (lost in processing) |

### Procedure:

i) Quetiapine fumarate, half portion each of Polyethylene oxide and Microcrystalline cellulose, and Hydroxyethyl cellulose were mixed together.
ii) Propylene glycol caprylate/caprate was dissolved in Isopropyl alcohol.
iii) Blend of step (i) was granulated with the solution of step (ii) and was passed through # 30 sieve.
iv) The granules of step (iii) was dried and mixed with remaining portion of Polyethylene oxide, Microcrystalline cellulose and Hydroxyethyl cellulose.
v) The mixture of step (iv) was filled into hard gelatin capsule.

### Example-8

| S. No. | Ingredient | mg/capsule |
|---|---|---|
| 1. | Eprosartan mesylate | 400.00 |
| 2. | Stearoyl macrogol glyceride | 50.00 |
| | (Gelucire ® 50/13) | |
| 3. | Mannitol | 40.50 |
| 4. | Xanthan gum | 220.00 |
| 5. | Hydroxypropyl methylcellulose | 100.00 |
| 6. | Dichloromethane (DCM) | q.s. (lost in processing) |

### Procedure:

i) Eprosartan mesylate, half portion of Xanthan gum and Hydroxypropyl methylcellulose were mixed together.
ii) Stearoyl macrogol glyceride was dissolved in Dichloromethane.
iii) Blend of step (i) was granulated with the solution of step (ii) and was passed through # 30 sieve.
iv) The granules of step (iii) was dried and mixed with remaining portion of Xanthan gum and Hydroxypropyl methylcellulose.
v) The blend of step (iv) was mixed with Mannitol.
vi) The mixture of step (v) was filled into hard gelatin capsule.

1. A novel sustained release pharmaceutical composition comprising at least one poorly soluble active agent(s), at least one solubilizer(s), a release rate controlling polymer system, and optionally other pharmaceutically acceptable excipients.

2. The composition as claimed in claim 1, wherein the composition additionally comprises at least one hydration inhibitor(s) present in an amount not less than about 5% by weight of the composition.

3. The composition as claimed in claim 1, wherein the release rate controlling polymer system comprises a combination of at least one acid soluble polymer and at least one pH independent polymer.

4. The composition as claimed in claim 3, wherein the ratio of the acid soluble polymer and the pH independent polymer is 1:50 to 50:1.

5. The composition as claimed in claim 2, wherein at least two hydration inhibitor(s) are present in a ratio of about 1:10 to about 10:1.

6. The composition as claimed in claim 1, wherein the active agent is selected from a group comprising cardiovascular drug, respiratory drug, sympathomimetic drug, cholinomimetic drug, adrenergic agonist, adrenergic antagonist, analgesic/antipyretic, anesthetic, antiasthamatic, antibiotic, antidepressant, antidiabetic, antifungal agent, antihypertensive agent, anti-inflammatory, antineoplastic, antianxiety agent, immunosuppressive agent, antimigraine agent, sedatives/hypnotic, antianginal agent, antipsychotic agent, antimanic agent, antiarrhythmic, antiarthritic agent, antigout agent, anticoagulant, thrombolytic agent, antifibrinolytic agent, hemorheologic agent, antiplatelet agent, anticonvulsant, antiparkinson agent, antihistamine/antipruritic, agent useful for calcium regulation, antibacterial agent, antiviral agent, antimicrobial, anti-infective, bronchodialator, hormone, hypoglycemic agent, hypolipidemic agent, protein, nucleic acid, agent useful for erythropoiesis stimulation, antiulcer/antireflux agent, antinauseant/antiemetic, oil-soluble vitamin, mitotane, visadine, halonitrosourea, anthrocycline or ellipticine and their pharmaceutically acceptable salts, esters, amides, polymorphs, solvates, hydrates, analogues, enantiomers, tautomeric forms or mixtures thereof, used either alone or in combination thereof.

7. The composition as claimed in claim 6, wherein the active agent is an antipsychotic agent.

8. The composition as claimed in claim 7, wherein the antipsychotic agent is selected from a group comprising emonaprode, diazepam, nitrazepam, flunitrazepam, lorazepam, prazepam, fluidiazepam, clonazepam, chlorpromazine hydrochloride, reserpine, clofluperol, trifluperidol, haloperidol, moperone, bromperidom, aripiprazole, sertindole, amisulpiride, asenapine, paloperidone or blonanserine, flupenthixol, fluphenazin, perphenazin, pimozide, chlorpromazine, tioridazine, melperone, zuclpentixol, etizolam, risperidone, olanzapine, clozapine, mipiprazole, quetiapine, ziprasidone, or its pharmaceutically acceptable salts, hydrates, polymorphs, esters, and derivatives thereof.

9. The composition as claimed in claim 8, wherein the active agent is ziprasidone or pharmaceutically acceptable salts, hydrates, polymorphs, esters, and derivatives thereof.

10. The composition as claimed in claim 9, wherein ziprasidone hydrochloride is present in substantially amorphous, semicrystalline or crystalline form

11. The composition as claimed in claim 9, wherein ziprasidone hydrochloride is in anhydrous or hydrated form or mixtures thereof.

12. The composition as claimed in claim 11, wherein ziprasidone hydrochloride is present as hemihydrate, monohydrate, dihydrate, trihydrate and tetrahydrate, or mixtures thereof.

13. The composition as claimed in claim 1, wherein the solubilizer is selected from a group comprising hydrophilic surfactants or lipophilic surfactants or mixtures thereof.

14. The composition as claimed in claim 13, wherein the solubilizer is selected from a group comprising PEG glyceryl stearate, PEG-40 hydrogenated castor oil, PEG 6 corn oil, lauryl macrogol-32 glyceride, stearoyl macrogol glyceride, polyglyceryl-10 dioleate, propylene glycol oleate, mono Propylene glycol dioctanoate, Propylene glycol caprylate/caprate, Glyceryl monooleate, Glycerol monolinoleate, PEG sorbitan monolaurate, PEG lauryl ether, Sucrose distearate, polyoxyethylene-polyoxypropylene block copolymer, polyethylene glycol hydroxystearate, Sodium lauryl sulphate, Sodium dodecyl sulphate, Dioctyl suphosuccinate, L-hydroxypropyl cellulose, hydroxylethylcellulose, hydroxy propylcellulose, Propylene glycol alginate, sodium taurocholate, sodium glycocholate, sodium deoxycholate, betains, polyethylene glycol, d-tocopheryl polyethylene glycol 1000 succinate and mixtures thereof.

15. The composition as claimed in claim 1, wherein the acid soluble polymer is selected from the group comprising polyalkylene oxides such as polyethylene oxide; cellulosic polymers such as methyl cellulose, hydroxypropyl cellulose, hydroxypropyl methylcellulose and hydroxyethyl cellulose; maleic anhydride polymers; poly(acrylamides); polyols; polyvinylamines; starch and starch-based polymers; polyurethane hydrogels; chitosan and its derivatives; polysaccharide gums; polyvinyl alcohol copolymers and the like or mixtures thereof.

16. The composition as claimed in claim 1, wherein the pH independent polymer is selected from a group comprising alkyl celluloses, hydroxyalkyl alkyl celluloses, hydroxy alkyl celluloses, polyethylene glycols, copolymers of ethylene oxide with propylene oxide, gelatin, polyvinylpyrrolidones, vinylpyrrolidones, vinyl acetates, polyvinylimidazoles, polyvinylpyridine N-oxides, copolymers of vinylpyrrolidone with long-chained alpha.-olefins, copolymers of vinylpyrrolidone with vinylimidazole, polyvinylpyrrolidone/dimethylaminoethyl methacrylates), copolymers of vinylpyrrolidone/dimethylaminopropyl methacrylamides, copolymers of vinylpyrrolidone/dimethylaminopropyl acrylamides, quaternised copolymers of vinylpyrrolidones and dimethylaminoethyl methacrylates, terpolymers of vinylcaprolactam/vinylpyrrolidone/dimethylaminoethyl methacrylates, copolymers of vinylpyrrolidone and methacrylamidopropyl-trimethylammonium chloride, terpolymers of caprolactam/vinylpyrrolidone/dimethylaminoethyl methacrylates, copolymers of styrene and acrylic acid, polycarboxylic acids, polyacrylamides, polyvinyl alcohols, hydrolysed polyvinyl acetate, copolymers of ethyl acrylate with methacrylate and methacrylic acid, copolymers of maleic acid with unsaturated hydrocarbons and mixed polymerisation products of the said polymers, polysaccharide gums, alginic acid, other alginates, benitonite, arabinoglactin, pectin, tragacanth, scleroglucan, dextran, amylose, amylopectin, dextrin, and the like or mixtures thereof.

17. The composition as claimed in claim 2, wherein the hydration inhibitor is selected from a group comprising stearic acid, glyceryl monostearate, glyceryl behenate, glyceryl monooleate, glyceryl palmitostearate, microcrystalline wax, stearyl alcohol, cetyl alcohol, cetostearyl alcohol, hydrogenated castor oil, tristearin, waxes, polyvinyl acetates, polyethylenes, polypropylenes, polyamides, ethylene glycol polyterephthalate, polyvinyl chlorides, polyformaldehyde chlorides, polycarbonates, ethylene copolymers, polyethers, polyurethanes, polyacrylonitriles, shellac, rosin, dicalcium phosphate and the like or mixtures thereof.

18. The composition as claimed in claim 1, wherein the pharmaceutically acceptable excipients are selected from a group comprising disintegrants, binders, mucoadhesive agents, fillers, bulking agents, anti-adherants, anti-oxidants, buffering agents, colorants, flavoring agents, coating agents, plasticizers, stabilizers, preservatives, lubricants, glidants, chelating agents, used either alone or in combination thereof.

19. A process of preparation of the composition as claimed in claim 1, which comprises of the following steps:
i) mixing the active agent(s) with solubilizer(s), and release rate controlling polymer system,
ii) optionally adding one or more other excipient(s), and
iii) formulating the mixture into a suitable dosage form.

20. A process of preparation of the composition as claimed in claim 1, which comprises of the following steps:
i) mixing the active agent(s) with other excipients and hydration inhibitor(s) and granulating with a solubilizer(s),
ii) mixing the granules of step (i) with the release rate controlling system,
iii) optionally adding one or more other excipient(s), and
iv) formulating the mixture into a suitable dosage form.

21. A process of preparation of the composition as claimed in claim 1, which comprises of the following steps:
i) mixing the active agent(s) with a portion of release rate controlling polymer system and hydration inhibitor(s) and granulating with a solubilizer(s),
ii) mixing the granules of step (i) with remaining portion of release rate controlling polymer system,
iii) optionally adding one or more other excipient(s), and
iv) formulating the mixture into a suitable dosage form.

22. A process of preparation of the composition as claimed in claim 1, which comprises of the following steps:
i) mixing the active agent(s) with other excipients,
ii) mixing the material of step (i) with the release rate controlling system,
iii) mixing the blend of step (ii) with hydration inhibitor(s) and other excipient(s),
iv) granulating the material of step (iii) with the solubilizer(s), and
v) formulating the mixture into a suitable dosage form.

23. (canceled)

24. (canceled)

25. (canceled)

26. (canceled)

27. A method for treating a disease, condition or symptom comprising administering to a subject in need thereof an effective amount of the composition according to claim 1.

28. A method for managing or treating psychosis or psychotic symptoms comprising administering to a subject in need thereof an effective amount of the composition according to claim 1.
